(19) United States
(12) Patent Application Publication
(10) Pub. No.: US 2009/0111769 A1
(43) Pub. Date: Apr. 30, 2009

(54) Antithrombotic Compound

(76) Inventor: Martin De Kort, Oss (NL)

Correspondence Address: ORGANON USA, INC.;c/o Schering-Plough Corporation, 2000 Galloping Hill Road, Mail Stop: K-6-1, 1990, Kenilworth, NJ, 07033, US

(21) Appl. No.: 12/083,213

(22) PCT Filed: Oct. 6, 2006
(86) PCT No.: PCT/EP2006/067128

§ 371 Date: Apr. 7, 2008

(30) Foreign Application Priority Data
Oct. 10, 2005 (EP) 05109403.5
Oct. 25, 2005 (EP) 05109963.8

(51) Int. Cl. A61K 31/715; C07H 15/20; A61P 7/02; C07H 1/00

(52) U.S. Cl. 514/54; 536/17.4; 536/18.6

(57) ABSTRACT

The present invention relates compounds of the formula I: oligosaccharide-spacer-GpIIb/IIIa antagonist I, wherein the oligosaccharide is a negatively charged pentasaccharide residue of the structure (I), the charge being compensated by positively charged counterions; the spacer is an essentially pharmacologically inactive linking residue of a length of 15-50 atoms; the GpIIb/IIIa antagonist is a residue derived from tirofiban or an analogue thereof; or a pharmaceutically acceptable salt thereof or a prodrug or a solvate thereof. The compounds of the invention have antithrombotic activity and can be used in treating or preventing thrombotic diseases.

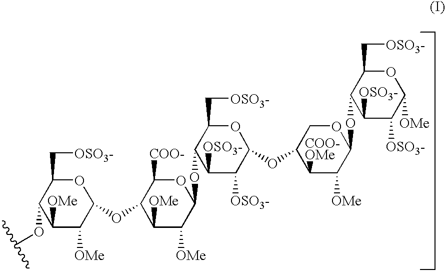

The invention relates to a new antithrombotic compound, a pharmaceutical composition containing the compound as an active ingredient, as well as the use of said compound for the manufacture of medicaments.

Acute myocardial infarction, ischemia and stroke are caused by the formation of an occlusive thrombus in an atherosclerotic coronary artery. The arterial thrombus is formed by blood platelets (thrombocytes) aggregating with (increased levels of) fibrinogen. This process is associated with an excited state and imbalance of the coagulation system in which fibrinogen is cleaved into fibrin clots. Intervention in one of these primary and secondary haemostatic pathways is essential in the treatment of (arterial) thrombosis.

Serine proteases are enzymes which play an important role in the blood coagulation cascade. Members of this group of proteases are for example thrombin, trypsin, factors VIa, IXa, Xa, XIa, XIIa, and protein C. Thrombin is the final serine protease enzyme in the coagulation cascade. The prime function of thrombin is the cleavage of fibrinogen to generate fibrin monomers, which are cross-linked to form an insoluble gel. In addition, thrombin regulates its own production by activation of factors V and VIII earlier in the cascade. It also has important actions at cellular level, where it acts on specific receptors to cause platelet aggregation, endothelial cell activation and fibroblast proliferation. Thus thrombin has a central regulatory role in haemostasis and thrombus formation. Factor Xa catalyzes the conversion of prothrombin into thrombin. Inhibition of factor Xa effectively results in inhibition of the coagulation of blood. Platelet aggregation is triggered by several activators, not only by thrombin, but also by ADP, collagen and epinephrin. In all cases, the final common pathway leading to platelet aggregation is binding of fibrinogen to its receptor, the key membrane glycoprotein complex GPIIb/IIIa. Therefore, inhibition of fibrinogen binding to this protein is considered a very effective way of inhibiting platelet aggregation for the prevention of (arterial) thrombus formation and the treatment of thrombotic disorders.

GPIIb/IIIa (α_{IIb}β₃) is a surface receptor belonging to the integrin family. Integrins are composed of two chains, an α subunit and a β subunit, which are held together by noncovalent bonds in a calcium dependent manner. GPIIb constitutes the α subunit (α_{IIB}) with divalent cation binding domains, whereas GPIIIa is a protypical β subunit (β₃). Integrins have been isolated from cells throughout the body and are mediators of cell-cell and cell-substrate adhesion and signalling. There are three binding sites on GPIIb/IIIa, one that recognises the amino sequence Arg-Gly-Asp (RGD binding site), another that recognises Lys-Gln-Ala-Gly-Asp (KQAGD binding site) and one that recognises Lys-Gly-Asp (KGD binding site).

On each circulating platelet, there are 35,000 to 100,000 GPIIb/IIIa complexes; most are distributed on the platelet surface, with a smaller pool in an internal reserve. The GPIIb/IIIa complex does not interact with its plasma ligands until platelets have been activated by exogenous agonists such as ADP or thrombin. When this occurs, an inside-out signal is generated that results in a conformational change in the extracellular portion of the complex that renders the molecule capable of binding fibrinogen and other ligands.

Compounds mimicking the α-chain (RGD) and γ-chain (KQAGDV) fragments of fibrinogen may act as antagonists. Numerous potent GPIIb/IIIa antagonists based on peptidomimetic structures have been described previously.

Some (very) potent examples are Ro 435054, xemilofiban, RWJ 50042, tirofiban and lamifiban. However, a significant number of other GPIIb/IIIa antagonists showing excellent potencies and pharmacological profiles in vitro, are not further developed or on hold after having reached late phase clinical trials, due to a lack of consistent control of platelet aggregation and ambiguous pharmacological behaviour, (partly) caused by the short half-lives of the compounds. The short half-lives lead to large variations in plasma levels of the free drug and may contribute to interindividual variability in dose response (monitoring therapy is required).

It was further reported by H. Darius in Thromb Res. 2001, 103, S117-S124 that in all large clinical trials with GPIIb/IIIa antagonists the therapeutic effect was only minor and, moreover, even an increased mortality in the glycoprotein IIb/IIIa receptor-antagonist-treated group of patients had been observed. The narrow therapeutic window and limited bioavailability of the drugs, together with the still very limited knowledge about the regulation of the platelet fibrinogen receptor, were considered to be responsible for this therapeutic failure.

In conclusion, there is a need for GPIIb/IIIa antagonists having a predictable antithrombotic effect, preferably with a longer half-life (to achieve consistent levels of inhibition of platelet aggregation).

In the European patent application 04005343.1 new compounds have now been reported which are dual inhibitors having a mixed pharmacological profile by inhibiting two key targets in both the coagulation cascade (factor Xa) and the platelet aggregation pathway (GpIIb/IIIa). The present invention relates to such dual inhibitors having particularly interesting pharmacological properties.

The compounds of this invention have the formula I

oligosaccharide-spacer-GpIIb/IIIa antagonist I,

wherein
the oligosaccharide is a negatively charged pentasaccharide residue of the structure

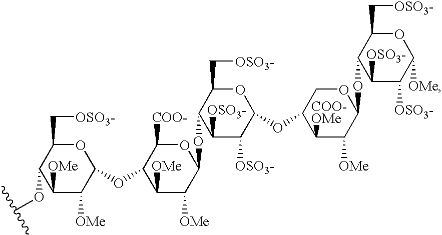

the charge being compensated by positively charged counterions;
the spacer is an essentially pharmacologically inactive linking residue of a length of 15-50 atoms;
the GpIIb/IIIa antagonist is a residue derived from tirofiban or an analogue thereof;
or a pharmaceutically acceptable salt thereof or a prodrug or a solvate thereof.

The compounds of the invention are effective antithrombotic agents by both ATIII-mediated inhibition of coagulation factor Xa and inhibition of platelet aggregation by antagonizing the binding of fibrinogen to its receptor. When compared to the combination therapies known in the art, wherein GpIIb/IIIa inhibitors are combined with anticoagulant therapies (such as described in Expert Opin. Investig. Drugs (2003) 12(9), 1567, in US 2003/0199457 A1 and U.S. Pat. No. 6,541,488), the pharmacokinetic and pharmacodynamic profiles of the compounds of the present invention lead to more consistent control of platelet aggregation and to less interindividual variability in dose reponse.

The compounds of the present invention are useful for treating and (possibly) preventing thrombotic diseases. This includes a number of thrombotic and prothrombotic states in which the coagulation cascade is activated which include, but are not limited to, deep vein thrombosis, pulmonary embolism, thrombophlebitis, arterial occlusion from thrombosis or embolism, arterial reocclusion during or after angioplasty, restenosis following arterial injury or invasive cardiological procedures, postoperative venous thrombosis or embolism, stroke and myocardial infarction.

In preferred embodiments the spacer is an essentially pharmacologically inactive linking residue having 25-35 atoms counted along the “backbone” of the spacer, the oxygen of the oligosaccharide residue not included. The chemical nature of the spacer is of minor importance for the anti-thrombotic activity of the compounds of the invention. The spacer may comprise (somewhat) rigid elements, such as ring structures and unsaturated bonds. However, the spacer of the compounds of the invention is preferably flexible. Suitable spacers may easily be designed by a person skilled in the art. For synthetic reasons longer spacers are considered less suitable, however, longer spacers may still successfully be applied in the compounds of the present invention. Preferred spacers contain at least one —(CH₂CH₂O)— element. More preferred spacers contain more, preferably six, —(CH₂CH₂O)— elements. The most preferred spacer is *—(CH₂CH₂O)₃—(CH₂)₂—NH—C(O)—CH₂O—(CH₂CH₂O)₃—(CH₂)₂—NH—C(O)—, the end indicated with * being attached to the oxygen of the oligosaccharide residue.

The attachment site of the spacer to the GpIIb/IIIa antagonist residue may be chosen essentially arbitrarily, provided that the GpIIb/IIIa antagonist activity is not abolished. Thus, the carboxylate moiety (optionally esterified) and the basic moiety of tirofiban (or an analogue thereof) must remain unaffected.

Preferably, the GpIIb/IIIa antagonist is a residue derived from an analogue of tirofiban.

An embodiment of the present invention is the compound having the structure II

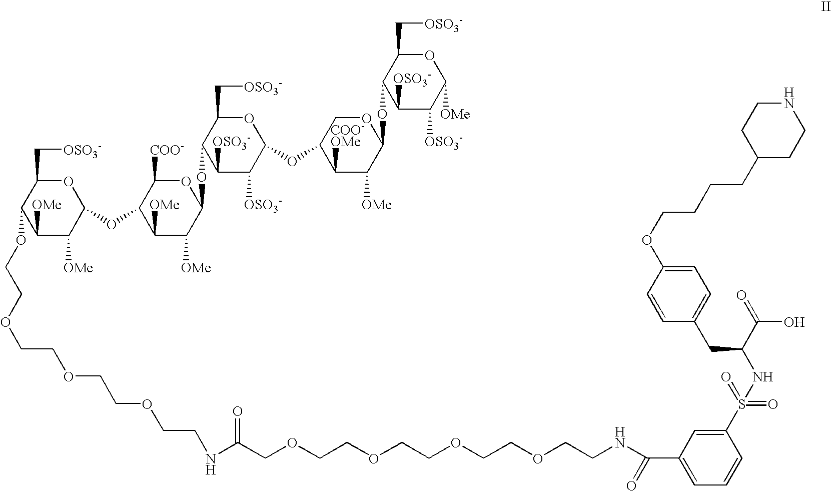

A positively charged counterion means H⁺, Na⁺, K⁺, Ca²⁺, and the like. Preferably the compounds of the invention are in the form of their sodium salt.

Tirofiban has the structure

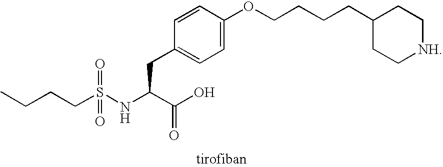

The term “prodrug” means a compound which is metabolized in the body into the active compound, e.g. compounds of formula I, wherein the carboxylate group in the GpIIb/IIIa antagonist residue is esterified.

Solvates according to the invention include hydrates.

The compounds of the present invention can be prepared by optionally modifying tirofiban with amino acids, peptidomimetics or additional functional groups (e.g. —COOH, —NH₂, —SH, —OH or the like) using methods generally known in the art. An example of the synthesis of such a modified compound is described in Bioorganic Chemistry 29, 357-379 (2001), where the compound is suggested as a potential vector for targeted drug delivery. According to the invention, the optionally modified tirofiban (a) is coupled to a pentasaccharide-spacer residue or (b) is coupled to a spacer, which subsequently is coupled to a pentasaccharide-spacer-residue (e.g. by methods known from WO 99/65934; WO 01/42262). The coupling of the spacer to the pentasaccharide can for instance be performed by using the methods described in EP 0,649,854.

The peptide coupling, a procedural step in the above described method to prepare the compounds of the invention, can be carried out by methods commonly known in the art for the coupling—or condensation—of peptide fragments such as by the azide method, mixed anhydride method, activated ester method, the carbodiimide method, or, preferably, under the influence of ammonium/uronium salts like TBTU, especially with the addition of catalytic and racemisation suppressing compounds like N-hydroxysuccinimide and N-hydroxybenzotriazole. Overviews are given in The Peptides, Analysis, Synthesis Biology, Vol 3, E. Gross and J. Meienhofer, eds. (Academic Press, New York, 1981) and Peptides: Chemistry and Biology, N. Sewald and H.-D. Jakubke (Wiley-VCH, Weinheim, 2002).

Amine functions present in the compounds may be protected during the synthetic procedure by an N-protecting group, which means a group commonly used in peptide chemistry for the protection of an α-amino group, like the tert-butyloxycarbonyl (Boc) group, the benzyloxycarbonyl (Z) group, the 9-fluorenylmethyloxycarbonyl (Fmoc) group or the phthaloyl (Phth) group, or may be introduced by demasking of an azide moiety. Overviews of amino protecting groups and methods for their removal is given in the above mentioned The Peptides, Analysis, Synthesis, Biology, Vol 3 and Peptides: Chemistry and Biology.

Amidine functions, if present, can be left unprotected in the coupling step, or can be protected using carbamate such as allyloxycarbonyl or benzyloxycarbonyl. The amidine function is preferably introduced under mild conditions by using the 1,2,4-oxadiazolin-5-one moiety as the precursor.

Carboxylic acid groups may be protected by a group commonly used in peptide chemistry for the protection of an α-carboxylic acid group, such as a tert-butyl ester. The carboxylic acid group of the modified GPIIb/IIIa antagonist is preferably protected as a benzyl ester. Removal of the protecting groups can take place in different ways, depending on the nature of those protecting groups. Usually deprotection takes place under acidic conditions and in the presence of scavengers or reductive conditions such as catalytic hydrogenation.

A prerequisite for conjugation of tirofiban (or an analogue thereof) to an oligosaccharide is the presence of an orthogonally reactive anchoring group, such as a carboxylate group, which can be coupled to an oligosaccharide-spacer derivative or via a spacer to an oligosaccharide-spacer derivative. To allow such conjugation in most cases (additional) modification of tirofiban is necessary.

Construction of the spacer-derived building blocks en route to compounds of the formula I can be achieved in various ways using methods known in the art, either in a linear fashion by the step-wise introduction of amino acids, their derivatives or peptidomimetics, or in convergent manner by block-coupling of intermediate constructs.

The compounds of the invention, which can occur in the form of a free base, may be isolated from the reaction mixture in the form of a pharmaceutically acceptable salt. The pharmaceutically acceptable salts may also be obtained by treating the free base of formula I with an organic or inorganic acid such as hydrogen chloride, hydrogen bromide, hydrogen iodide, sulfuric acid, phosphoric acid, acetic acid, propionic acid, glycolic acid, maleic acid, malonic acid, methanesulphonic acid, fumaric acid, succinic acid, tartaric acid, citric acid, benzoic acid, ascorbic acid and the like.

The compounds of this invention or intermediates thereof may possess chiral carbon atoms, and may therefore be obtained as a pure enantiomer, or as a mixture of enantiomers, or as a mixture containing diastereomers. Methods for obtaining the pure enantiomers are well known in the art, e.g. crystallization of salts which are obtained from optically active acids and the racemic mixture, or chromatography using chiral columns. For diastereomers straight phase or reversed phase columns may be used.

The compounds of the invention may be administered enterally or parenterally. The exact dose and regimen of these compounds and compositions thereof will necessarily be dependent upon the needs of the individual subject to whom the medicament is being administered, the degree of affliction or need and the judgement of the medical practitioner. In general parenteral administration requires lower dosages than other methods of administration which are more dependent upon absorption. However, the daily dosages are for humans preferably 0.0001-10 mg per kg body weight, more preferably 0.001-1 mg per kg body weight.

The medicament manufactured with the compounds of this invention may also be used as adjuvant in (acute) anticoagulant therapy. In such a case, the medicament is administered with other compounds useful in treating such disease states, such as aspirin, clopidogrel or statins. Mixed with pharmaceutically suitable auxiliaries, e.g. as described in the standard reference, Gennaro et al., Remington's Pharmaceutical Sciences, (18th ed., Mack Publishing Company, 1990, see especially Part 8: Pharmaceutical Preparations and Their Manufacture) the compounds may be compressed into solid dosage units, such as pills, tablets, or be processed into capsules or suppositories. By means of pharmaceutically suitable liquids the compounds can also be applied in the form of a solution, suspension, emulsion, e.g. for use as an injection preparation, or as a spray, e.g. for use as a nasal spray.

For making dosage units, e.g. tablets, the use of conventional additives such as fillers, colorants, polymeric binders and the like is contemplated. In general any pharmaceutically acceptable additive which does not interfere with the function of the active compounds can be used.

Suitable carriers with which the compositions can be administered include lactose, starch, cellulose derivatives and the like, or mixtures thereof, used in suitable amounts.

The invention is further illustrated by the following examples.

### EXAMPLES

### Abbreviations Used

ADP=adenosine diphosphate

ATIII=antithrombin III

Bn=benzyl

DiPEA=N,N-diisopropylethylamine

DMF=N,N-dimethylformamide

Et=ethyl

Me=methyl

RT=room temperature

TBTU=2-(1H-benzotriazol-1-yl)-1,1,3,3-tetramethyluronium tetrafluoroborate

TRAP=thrombin receptor agonist peptide

z=benzyloxycarbonyl

### Example 1

### Methyl O-2,3-di-O-methyl-4-O-<<12-N-<3-{[15-N-(15-aza-1-keto-3,6,9,12-tetraoxapentadecyl)]-carbonyl}-benzenesulfonyl>-4-O-{4-(4-piperidinyl)-butyl}-L-tyrosyl>-12-aza-3,6,9-trioxa-dodecyl>>-6-O-sulfo-alpha-D-glucopyranosyl-(1->4)-O-2,3-di-O-methyl-beta-D-glucopyranuronosyl-(1->4)-O-2,3,6-tri-O-sulfo-alpha-D-glucopyranosyl-(1->4)-O-2,3-di-O-methyl-alpha-L-idopyranuronosyl-(1->4)-2,3,6-tri-O-sulfo-alpha-D-glucopyranoside nonakis sodium salt (3)

### (Scheme 1)

Compound 3 was obtained by coupling of compound 1 (102 mg, 54 μmol)) [which may be obtained by coupling of the derivatised monosaccharide 5 described in WO 01/42262 with the tetrasaccharide 48 described in US 2004/0024197 using methods similar to those described in these patent applications, including deprotection and sulfation] with compound 2 (60 mg, 57 μmol), which was prepared as described in EP 04005343.1.

Thus, the carboxylic acid derivative (57 μmol) 2 was dried by repeated concentration in dry DMF (2×3 mL), dissolved in DMF (2 mL) and stirred in the presence of TBTU (19 mg, 57 μmol) and DiPEA (9.8 μL, 57 μmol), under an atmosphere of N₂. After 1 h, pentasaccharide 1 (53 μmol) was added. The reaction mixture was stirred overnight at RT and analyzed by ion exchange (Mono-Q) and reversed phase (Luna C18) chromatography. The reaction mixture was concentrated (<50° C., 15 mmHg). The (crude) product (10 mg/mL in H₂O/t-BuOH, 1/1, v/v) was deprotected by hydrogenation (H₂) over 10% Pd/C (an equal amount in weight was added with respect to the crude product). After 16 h the solution was degassed, filtered over a 0.45 μM HPLC filter and concentrated under reduced pressure (<50° C., 15 mmHg). The conjugate was purified by ion exchange chromatography (Q-sepharose, buffer: H₂O→2M NaCl), followed by desalting with a Sephadex G25-column (H₂O) and lyophilization. Yield 72 mg (52%).

¹H-NMR (D₂O, 600 MHz, HH—COSY): δ 7.84 (m, 1H), 7.75 (m, 1H), 7.62 (m, 1H), 7.42 (t, 1H), 6.83 (d, 2H), 6.48 (d, 2H), 5.38 (d, 1H), 5.33 (m, 1H), 5.08 (d, 1H), 5.02 (bs, 1H), 4.58 (d, 1H), 4.46 (bs, 2H), 4.28 (m, 2H), 4.17 (m, 1H), 4.22 (m, 1H), 4.20 (m, 2H), 4.11 (m, 2H), 4.04 (d, 1H), 4.03 (m, 1H), 4.02 (m, 1H), 3.88 (m, 2H), 3.84 (m, 2H), 3.82 (m, 1H), 3.79 (m, 1H), 3.72 (1H, m), 3.66 (m, 2H), 3.62-3.33 (m, 54H), 3.20 (dd, 1H), 3.18 (m, 1H), 2.92 (m, 2H), 1.92 (m, 2H), 1.70 (m, 2H), 1.58 (m, 1H), 1.44 (m, 2H), 1.33 (m, 4H).

ESI-MS: m/z 1225.2 [M+5H+2Na]²⁻, 823.8 [M+3Na⁺ 3H]³⁻, 816.5 [M+2Na+4H]³⁻, 809.1 [M+Na+5H]³⁻.

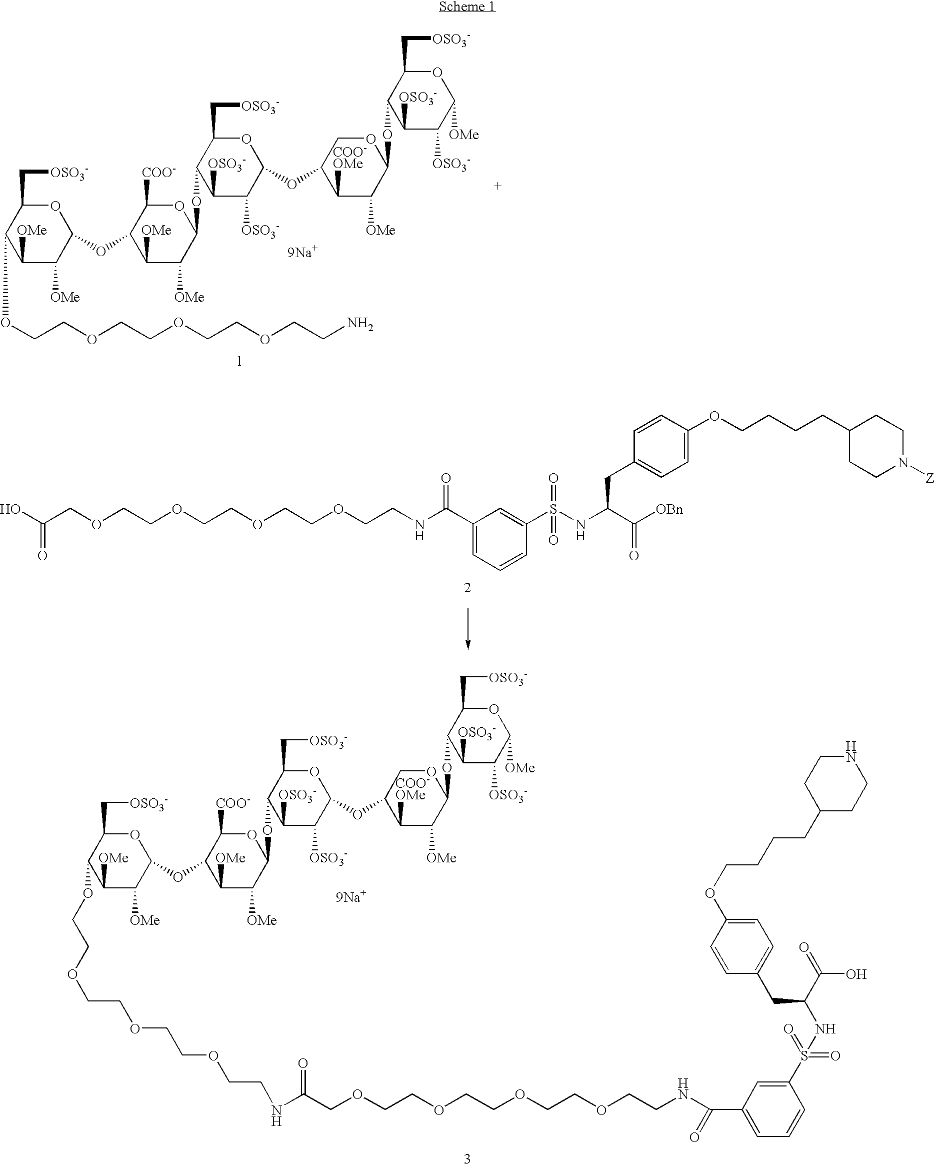

### Example 2

### Pharmacology

### 1.1 In Vitro Test for Inhibition of Guinea Pig Platelet Aggregation Induced by ADP

Addition of adenosine diphosphate (ADP) to human or guinea pig platelet rich plasma (PRP) in vitro induces platelet aggregation. This aggregation can be assessed by measuring the change in optical density (OD) of the PRP. The following in vitro test was used to evaluate the test compound for interference with the ADP-induced aggregation of guinea pigs.

### Materials

Platelet rich plasma (PRP): Free-flowing blood is taken from a healthy volunteer or a guinea pig and collected in 0.1 volume sodium citrate.2H₂O, 3.8% in distilled H₂O (w/v). The final concentration is 0.38% sodium citrate. The citrated blood is centrifuged at 1,600 N/kg (160 g, i.e., 900 rpm in a Hettich Rotanta/AP at room temperature. After 15 minutes centrifugation is discontinued with brake turned off, and the supernatant (=PRP) is collected. A fresh solution of ADP (analytical grade), 50 μM in 0.9% NaCl in MQ water, is used immediately.

In this assay, tirofiban (AGGRASTAT® (MSD) purchased as 0.25 mg/mL concentrate for i.v. infusion) inhibits human platelet aggregation induced by 5 μM ADP by 50% at a concentration of 30-60 nM (IC50).

### Equipment

1. Sysmex blood cell counter model KX-21.
2. Labsystems iEMS reader MF with a 620 nm filter, an orbital shaker set at 1000 rpm and a constant temperature of 37° C. Absorption is measured with the Labsystems iEMS program.
3. Blood collection system 600 mL with needle, art P4203 (NPBI).
4. 96 wells flat bottom microplates (Greiner Labortechnik).

### Procedure

The platelets in the supernatant (PRP) are counted using a Sysmex blood cell counter and the supernatant is diluted with PPP (platelet poor plasma) to obtain a PRP containing approximately 400,000±50,000 plt/mL. PRP should stabilize at room temperature for at least 20 min but not longer than 3 h.

150 μL PRP is pipetted into a well of the microplate. 30 μL test compound in a range of concentrations (7 concentrations per compound) or vehicle is added and the microplate is placed in the Labsystems iEMS reader MF at 37° C. The optical density (OD620) is then measured at 620 nm. After shaking for 2 minutes in the reader (1,000 rpm), the OD is measured for the second time. This is to verify the stability of the platelets (absence of spontaneous platelet aggregation). Then, 20 μL of 50 μM ADP solution is added and the OD is kinetically measured every minute for 14 minutes at 620. Between two measurements, the plate is shaking for 40 seconds at 1,000 rpm. Each test compound is investigated in at least 2 experiments using PRP from different volunteers.

### Evaluation of Response

The mean OD at each compound concentration (including vehicle) is calculated at t=0 min and t=10 min. The percentage inhibition at each concentration is calculated using the formula:

$$100 \% - \frac{( ODcompound at t = 0 min - ODcompound at t = 10 min )}{( ODvehicle at t = 0 min - ODvehicle at t = 10 min )} \times 100 \%$$

The IC50 of the test compound is the concentration at which the ADP-induced platelet aggregation is reduced by 50%. For this, the percentage inhibition values are plotted against compound concentration and the IC50 is calculated using Graphpad Prism 3.0 (with variable slope).

### 1.2 In Vitro Test for Inhibition of Human Platelet Aggregation Induced by TRAP

Addition of trombin receptor agonist peptide (TRAP) to washed human or guinea pig platelets (WPL) in vitro induces platelet aggregation. This aggregation can be determined by measuring the optical density of the WPL. The in vitro test described here is used to analyze the activity of a test compound to inhibit TRAP-induced aggregation of human platelets. A microplate reader is used to measure the activity of several compounds simultaneously.

### Materials

### Composition of Watson Buffer:

| | |
|---|---|
| NaCl | 7.83 g (134 mmol) |
| KCl | 0.22 g (2.9 mmol) |
| NaHCO₃ | 1.01 g (12 mmol) |
| Na₂HPO₄•2H₂O | 0.06 g (0.34 mmol) |
| MgCl₂•6H₂O | 0.20 g (1 mmol) |
| Glucose | 0.90 g (5 mmol) |
| HEPES | 1.19 g (5 mmol) |
| H₂O to | 1 L |

The pH is adjusted to 7.4 with NaOH (1 mol/L) TNP buffer.

### Composition of TNP-Buffer:

| | |
|---|---|
| Tromethamine (Tris) | 6.057 g (50 mmol) |
| NaCl | 5.844 g (100 mmol) |
| PEG6000 | 3.0 g |
| H₂O to | 1 L |

The pH of the solution is adjusted to 7.4 at 37° C. with HCl (10 mol/L).

### PGI₂ Solution:

Prostaglandin I₂ stock solution of 1 mg/mL in KOH (1 mol/L) is stored at −20° C. Immediately before use a solution of 5 μg/mL in ice-cold NaCl (9.0 g/L) is prepared.

### Platelet Rich Plasma (PRP):

Free-flowing blood is taken from a healthy volunteer or a guinea pig and collected in 0.1 volume 3.8% sodium citrate.2H2O in MQ water (w/v). The final conncentration is 0.38% sodium citrate. The citrated blood is centrifuged at 1,600 N/kg (160 g) in Hettich Rotanta/AP centrifuge at room temperature. After 15 minutes, centrifugation is discontinued with the brake turned off. And the supernatant (=PRP) is collected and diluted with platelet poor plasma to obtain a suspension containing approximately 400 000 platelets/mL.

### Platelet Poor Plasma (PPP):

Citrated blood is centrifuged at approximately 20000 N/kg for 10 minutes at RT and the PPP is siphoned off.

### Washed Platelets (WPL):

An aliquot of 1 μL PGI₂ solution is added to 1 mL PRP and thereafter centrifuged at 20000 N/kg for 10 minutes at RT. The plasma is siphoned off and Watson buffer containing 5 ng/mL PGI₂ is added to the platelet pellet and the platelets are resuspended in the original volume by gently stirring with a plastic rod. The platelet suspension is centrifuged again at 20000 N/kg. The platelets are resuspended in Watson buffer in order to obtain a suspension containing approximately 400 000 platelets/mL.

### TRAP Solution:

TRAP is dissolved in H₂O to give a solution containing 50 μmol/L. A fresh solution has to be prepared daily. For all aqueous solutions ultrapure H₂O (Milli-Q quality) is used.

Human Fibrinogen (Kordia/ERL, art nr: FIB 2 powder): 0.5 g fibrinogen powder is dissolved in 50 mL MQ water under vacuum. This stock solution is stored in aliquots of 100 μL at −20° C. Immediately before use, a solution of 0.5 mg/mL in saline is prepared.

In this assay, tirofiban (AGGRASTAT® (MSD)) purchased as 0.25 mg/mL concentrate for i.v. infusion) inhibits the human platelet aggregation induced by 5 μM TRAP by 50% at a final concentration of 30-60 nM (IC50).

### Procedure

The WPL concentration is counted in a Sysmex blood cell counter and the suspension is diluted with Watson buffer to obtain a concentration of approximately 400,000 plt/mL. Before use WPL is allowed to stabilize at room temperature for at least 20 min but not longer than 3-4 hours.

150 μL WPL is pipetted into a well of a microplate. 15 μL of test compound solution or vehicle and 15 μL fibrinogen solution is added and the microplate is placed in the microplate reader at 37° C. Then, the optical density (OD) is measured at 405 nm and after shaking for 2 minutes in the reader, the OD405 is measured again to verify the stability of the platelets (absence of spontaneous platelet aggregation). 20 μL of 50 μM TRAP solution is added and the OD405 is kinetically measured every min for 14 min at 405 nm. Between two measurements, the plate is shaking for 40 seconds at 1,000 rpm. For determination of the IC₅₀ of a test compound, each compound is investigated in at least 2 experiments using WPL from different volunteers.

### Evaluation of Responses:

The mean OD of each concentration (including vehicle) is calculated at t=0 min and t=10 min. The percentage inhibition at each concentration is calculated using Microsoft Excel with the formula:

$$100 \% - \frac{( ODcompound at t = 0 min - ODcompound at t = 10 min )}{( ODvehicle at t = 0 min - ODvehicle at t = 10 min )} \times 100 \%$$

The concentrations of the compound are plotted against the percentage inhibition. The IC₅₀ is calculated using Graphpad Prism 3.0 (with variable slope). The IC₅₀ of the test compound is the concentration at which the TRAP-induced platelet aggregation is reduced by 50%.

### 1.3 In Vitro Test for Determination of the Anti-Factor Xa Activity in Human Plasma

The anti-factor Xa activity of the tested compounds in human plasma were measured amidolytically with S2222 (Chromogenix, Chromogenics Ltd, Molndal, Sweden) using the method described by Teien and Lie. (Teien A N, Lie M. Evaluation of an amidolytic heparin assay method increased sensitivity by adding purified antithrombin III. Thromb. Res. 1977, 10: 399-410). The anti-Xa activity is expressed in U/μmol after comparison of the amidolytic activity with a calibration curve of standard heparin.

TABLE 1
Summary of in vitro antithrombotic activities

| | Anti-Xa | Inhibition of | Inhibition of | Inhibition of |
|---|---|---|---|---|
| | U/umol | human | human | guinea pig |
| | Human | platelet | platelet | platelet |
| | plasma | aggregation | aggregation | aggregation |
| Compound | pH 7.4 | (ADP) | (TRAP) | (ADP) |
| 3 | 1351 | 92 | 65 | 127 |

### 2.1 Pharmacokinetics

The pharmacokinetic properties of compound 3 were studied in male Wistar rats of 300-400 gr. The rats were anaesthetized by inhalation of a mixture of O₂/N₂O/isoflurane, after which the right jugular vein was cannulated. The next day rats were treated s.c. with doses of 100 or 500 nmol/kg. After s.c. administration, blood was sampled at several time intervals. Then the blood was centrifuged after which the plasma was siphoned off and stored at −20° C. until use. The concentration of the tested compound was measured amidolytically with S2222 (Chromogenix, Chromogenics Ltd, Molndal, Sweden) by determination of the anti-Xa activity based on the method of Teien and Lie in the obtained plasma samples against a calibration curve which was made of the stock solution of the tested compound itself. (Teien A N, Lie M. Evaluation of an amidolytic heparin assay method increased sensitivity by adding purified antithrombin III. Thromb. Res. 1977, 10: 399-410). The concentration in the samples was expressed in nmol/mL and the kinetic parameters were calculated with the noncompartment model of WinNonlin.

TABLE 2
Pharmacokinetic parameters after s.c. administration of compound 3
(500 nmol/kg) in rat. Experiment performed in n = 3/treatment.

| | Compound 3 |
|---|---|
| | Mean ± s.e.m. |
| Tmax (h) | 2.5 |
| Cmax (nmol/mL) | 5.0 ± 0.4 |
| T½ eli (h) | 9.3 ± 0.2 |
| AUCinf (h · nmol/mL) | 75.3 ± 3.2 |
| Vz (mL/kg) | 90 ± 5 |
| Cl (mL/h/kg) | 6.7 ± 0.3 |

1-10. (canceled)

11. A compound of the formula I
oligosaccharide-spacer-GpIIb/IIIa antagonist I,
wherein
the oligosaccharide is a negatively charged pentasaccharide residue of the structure 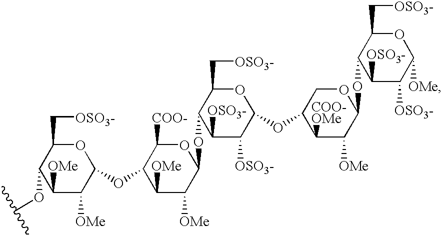
the charge being compensated by positively charged counterions;
the spacer is an essentially pharmacologically inactive linking residue of a length of 15-50 atoms;
the GpIIb/IIIa antagonist is a residue derived from tirofiban or an analogue thereof;
or a pharmaceutically acceptable salt thereof or a prodrug or a solvate thereof.

12. The compound of claim 11, wherein the spacer has a length of 25-35 atoms.

13. The compound of claim 11, wherein the spacer comprises at least one —(CH₂CH₂O)— element.

14. The compound of claim 11, wherein the GpIIb/IIIa antagonist is a residue derived from an analogue of tirofiban.

15. The compound of claim 11, having the structure

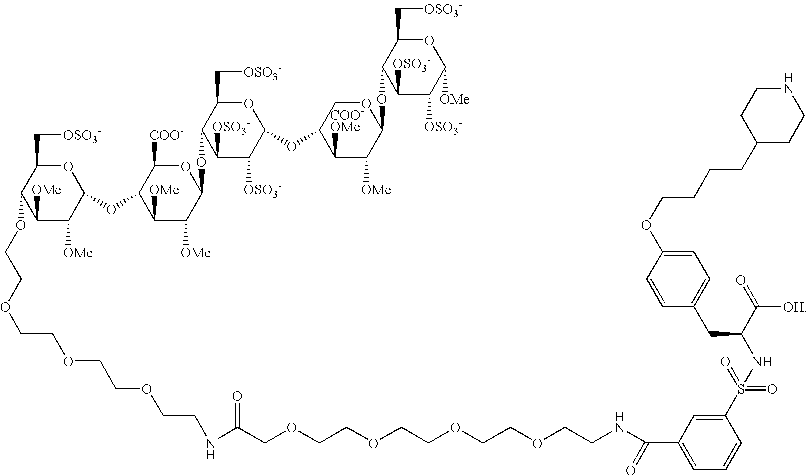

16. The compound of claim 15, being in the form of its sodium salt.

17. A process for the preparation of the compound of formula I, comprising a step wherein a modified tirofiban analogue is coupled to a spacer, which is subsequently coupled to an oligosaccharide-spacer-residue.

18. A pharmaceutical composition comprising the compound of claim 11 and pharmaceutically suitable auxiliaries.

19. A pharmaceutical composition comprising the compound of claim 15 and pharmaceutically suitable auxiliaries.

20. A method for treating or preventing thrombosis or other thrombin-related diseases in a patient, the method comprising administering to the patient an effective amount of the compound according to claim 11.
